(19) United States
(12) Patent Application Publication
(10) Pub. No.: US 2020/0069522 A1
(43) Pub. Date: Mar. 5, 2020

(54) POLYMERIC CONTAINER FOR DOSING OF SOLID PRODUCTS CONTAINING ACTIVE INGREDIENTS

(71) Applicant: Honey Forms LLC, Northglenn, CO (US)

(72) Inventor: Jason D. LUTZ, Superior, CO (US)

(21) Appl. No.: 16/559,494

(22) Filed: Sep. 3, 2019

Related U.S. Application Data
(60) Provisional application No. 62/726,031, filed on Aug. 31, 2018.

(51) Int. Cl. A61J 3/00; B65D 1/24; B01D 9/00; A61K 31/352

(52) U.S. Cl. CPC A61J 3/00; B65D 1/24; B01D 2009/0086; A61K 31/352; B01D 9/0018

(57) ABSTRACT

Disclosed herein are devices and methods useful in apportioning a liquid product into solid doses, wherein individual doses comprise equivalent amounts of an active ingredient. In an exemplary embodiment, a sealable container with integrated multiple cells for dosing of small amounts of dried concentrated crystalline extract is described. The containers allow for ease in transport, shipment, storage, and apportionment/dispensing of the dried extract. Different containers may have various sizes and shapes of cells, however, each cell in a given container holds and/or defines a similar amount of dried extract. The container may further include a cap or lid structure for securing the container and containing the dried extract. In most cases, the container is manufactured from a heat resistant, food-grade, pliable polymer to aid in dispensing individual doses of the dried extract.

### CROSS REFERENCE TO RELATED APPLICATIONS

This application claims the benefit of U.S. Provisional Application No. 62/726,031, filed 31 Aug. 2018, and entitled “Polymeric container for dosing of solid products containing active ingredients,” the entirety of which is incorporated herein by reference for all purposes.

### TECHNICAL FIELD

The technology described herein relates generally to devices, compositions, and methods for use in apportioning doses of solid products containing active ingredients.

### BACKGROUND

Multi-cell molded plastic trays are useful in providing for discrete portioned amounts of a product. For example, ice trays are useful in creating ice cubes, and molded trays have found use in preparation of baked goods. Such items have not been found to be of use in the medicinal or herbal industries, which often require more concentrate and/or smaller portions.

Dosing of portions in medicine, including herbal medicine, requires, in many cases, that the product be liquid to allow for control of active ingredients. However, not all active ingredients are able to be dissolved or maintained in liquid form. In addition, liquid medicines or remedies are not always preferred as it may be more difficult to apportion small doses in liquid form. This may be especially true where the medicine or remedy may be administered in volatilized or vaporized form.

Applicants describe herein devices, systems, and methods that meet the present needs.

### SUMMARY

Disclosed herein are devices, compositions, and methods for use in maintaining the viability of therapeutic mammalian cells at low temperatures, the disclosed devices are also useful in reducing manipulation of cells prior to administration to a patient in need thereof. The disclosed compositions, devices, and methods maintain the cells' viability to help reduce loss of therapeutic effect.

In several embodiments, a polymeric container for apportioning an active ingredient is disclosed. The container may include a cavity defined by a cavity floor and a plurality of cavity walls. Two or more cells may be positioned within the cavity. The cells may include a cell floor and at least one cell divider extending from the cell floor to a cell divider top. Each cell of the two or more cells may define a volume defined by a cross-sectional area of the respective cell and the distance from the cell floor to the cell divider top of the respective cell.

In several embodiments, a method of apportioning a liquid is disclosed. The method includes adding a volume of a liquid to a polymeric container having a cavity defined by a container floor and a plurality of container walls, and two or more cells positioned within the cavity, the cells comprising a cell floor and at least one cell divider extending from the cell floor to a cell divider top, wherein each cell defines a volume defined by a cross-sectional area of the respective cell and the distance from the cell floor to the cell divider top of the respective cell, wherein the volume of the liquid is greater than the sum of the volumes defined by the two or more cells; allowing the liquid to form a planar surface generally positioned further from the cell floor than the level of the cell wall divider; treating the liquid in the container to aid in transitioning the liquid to a solid; and allowing the liquid to form a solid at room temperature.

In several embodiments, a container for apportioning equivalent doses of an active ingredient is disclosed. The container may include a plurality of primary cells, the primary cells defined by a primary cell wall and a primary cell floor, wherein each primary cell defines a first shape and an equivalent first volume, and adjacent primary cells are separated by a primary cell divider. The container may include a plurality of secondary cells, the secondary cells defined by a secondary cell wall and a secondary cell floor, wherein each secondary cell defines a second shape and an equivalent second volume, and adjacent secondary cells are separated by a secondary cell divider. The plurality of primary cells may be positioned within the plurality of secondary cells and the secondary cell divider may have a height greater than the primary cell divider.

Additional objects and advantages of the invention will be set forth, in part, in the description which follows and, in part, will be obvious from the description, or may be learned by practice of the invention. The objects and advantages of the invention will be realized and attained by means of the elements and combinations, particularly pointed out in the appended claims.

### BRIEF DESCRIPTION OF THE DRAWINGS

FIG. 1 is a perspective view of one embodiment of a dosing container.

FIG. 2 is a perspective side cross-sectional view of the container in accordance with the embodiment of the container illustrated in FIG. 1 taken along section line I-I.

FIG. 3 is a bottom elevation view of the container in accordance with the embodiment of the container illustrated in FIG. 1.

FIG. 4 is a top elevation view of the container in accordance with the embodiment of the container illustrated in FIG. 1.

FIG. 5 is a side view of the container in accordance with the embodiment of the container illustrated in FIG. 1.

FIG. 6 is an exploded view of the container having a cap of in accordance with the embodiment of the container illustrated in FIG. 1.

FIG. 7 is a perspective view of another embodiment of a dosing container.

FIG. 8 is a perspective view of another embodiment of a dosing container.

FIG. 9 is a side view of the container in accordance with the embodiment of the container illustrated in FIG. 8.

FIG. 10 is a top elevation view of the container in accordance with the embodiment of the container illustrated in FIG. 8.

FIG. 11 is a bottom elevation view of the container in accordance with the embodiment of the container illustrated in FIG. 8.

FIG. 12 is a perspective view of another embodiment of a dosing container.

FIG. 13 is a side view of the container in accordance with the embodiment of the container illustrated in FIG. 12.

FIG. 14 is a top elevation view of the container in accordance with the embodiment of the container illustrated in FIG. 12.

FIG. 15 is a bottom elevation view of the container in accordance with the embodiment of the container illustrated in FIG. 12.

FIG. 16 is a perspective view of another embodiment of a dosing container.

FIG. 17 is a side view of the container in accordance with the embodiment of the container illustrated in FIG. 16.

FIG. 18 is a top elevation view of the container in accordance with the embodiment of the container illustrated in FIG. 16.

FIG. 19 is a bottom elevation view of the container in accordance with the embodiment of the container illustrated in FIG. 16.

FIG. 20 is a perspective view of another embodiment of a dosing container.

FIG. 21 is a side view of the container in accordance with the embodiment of the container illustrated in FIG. 20.

FIG. 22 is a top elevation view of the container in accordance with the embodiment of the container illustrated in FIG. 20.

FIG. 23 is a bottom elevation view of the container in accordance with the embodiment of the container illustrated in FIG. 20.

FIG. 24 is a perspective view of another embodiment of a dosing container.

FIGS. 25 and 26 are opposing side views of the container in accordance with the embodiment of the container illustrated in FIG. 24.

FIGS. 27 and 28 are opposing front and back views of the container in accordance with the embodiment of the container illustrated in FIG. 24.

FIG. 29 is a top elevation view of the container in accordance with the embodiment of the container illustrated in FIG. 24.

FIG. 30 is a bottom elevation view of the container in accordance with the embodiment of the container illustrated in FIG. 24.

### DETAILED DESCRIPTION

The disclosed containers are useful in the dosing, storage, and transportation of dried extracts, for example shatter, resin, or wax. In most embodiments, liquid extract may be dispensed into the described containers and dried in place. After drying, the extract becomes brittle. The brittle solid product may have a generally flat first surface and a second, opposite surface that reflects the cavity structures of a plurality of cells formed in the bottom or cavity floor of the container. This creates a plurality of solid product in the shape of the cell's negative space. This scoring results in the ability to separate the shatter into multiple, uniform individual doses. While the doses may have different shapes, for example where the cells are not uniform, they will generally comprise substantially the same amount of active ingredient. The resulting similarity in amount of active ingredients permits the user to select the number of shatter doses and amount of active ingredient without having to weigh variously sized random fragments to determine the amount of active ingredient desired.

Molded polymeric containers having a plurality of individual cells or compartments are described. The shapes of the container and of individual cells within each container may vary, but each individual cell from a given container may describe a volume within and above the cell that is substantially equivalent to the other cells of the container.

Shatter is a term used to describe concentrated, dried extract. In most cases, the extract is herbal and derived from a botanical source including plants, leaves, flowers, etc. In some cases, shatter may refer to a cannabinoid-containing concentrate. The texture and form of the concentrate results in its breakage, or shattering, like glass.

Shatter may be manufactured by first extracting the botanical source. In some cases, the botanical source may first be dried, and then subjected to extraction with various solvents—for example butane, chloroform, dichloromethane, petroleum ether, naphtha, benzene, methanol, ethanol, isopropanol, etc. This results in an oil that is then vacuum dried to remove the solvent and other volatile compounds. Shatter is usually translucent and may have a generally amber color.

Shatter, resin, and wax are generally purchased and/or dispensed by weighing randomly shaped pieces or shards, until the desired amount is obtained. Thus, an individual user, wishing to consume a fraction of the dispensed amount, must reweigh fragments of the original purchase until the desired amount is achieved. Alternatively, if the user does not have access to a meter, the user must estimate the amount desired by selecting among the random pieces or shards.

As provided in more detail herein, a container 100 may have a plurality of cells 130 that define either generally uniform volumes or a plurality of identifiable volumes. The disclosed container 100, having a plurality of cells 130 defining a plurality of identifiable volumes (e.g., either generally uniform or variable), addresses the difficulty faced by users of shatter, wax, and resin, by creating easily separable, doses having generally identifiable amounts of an active ingredient in the product, whether equivalent or variable. In some embodiments, the container 100 may comprise between 5 and 100 cells 130. In many embodiments, the container 100 may comprise between about 10 and 30 cells 130. The cells 130 may be randomly shaped, or uniformly shaped. In various embodiments, the cell shape may have between three and 10 sides. In many embodiments, the shape of the cells is selected from triangular, square, hexagonal, and octagonal.

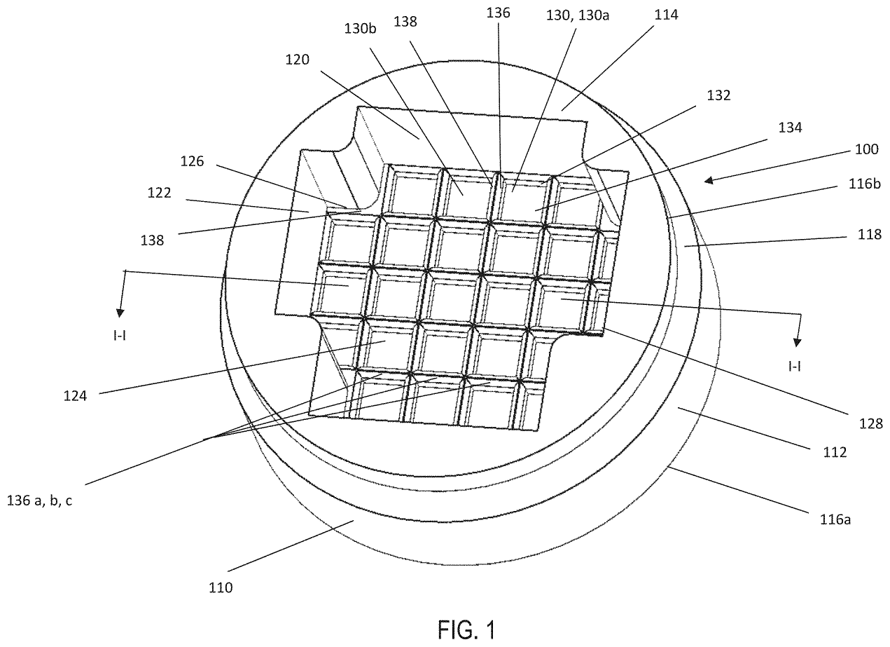

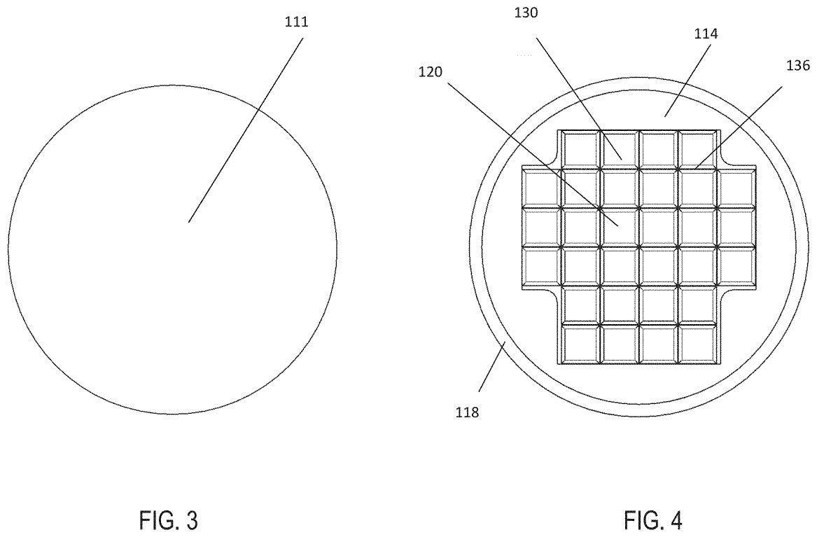

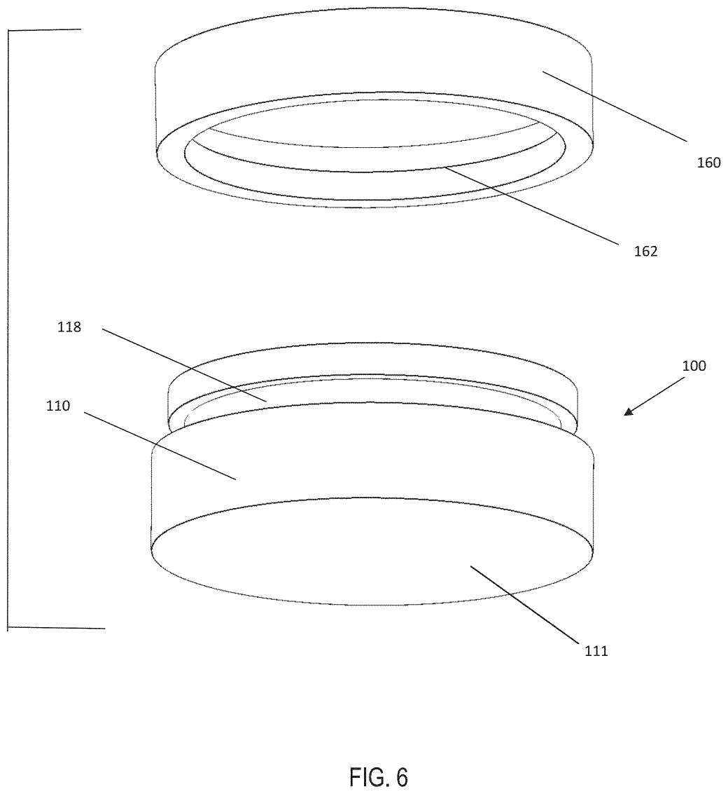

The container 100 may be of various shapes, having an exterior or outer surface 112. The outer surface 112 may extend from a base or bottom edge 116a to an upper or top edge 116b. In most embodiments, the underside of the container 100 may define a bottom surface 111 (See FIGS. 3 and 6) for use in resting the container 100 on a receiving surface (not shown in FIG. 1), such as a table top or the palm of a user. In most embodiments, the bottom surface 111 may be generally planar. At or near the top edge 116b may be an upper or top surface 114. Like the bottom surface 111, the top surface 114 may be generally planar, but may define an indentation or cavity 120. In various examples, the cavity 120 may be positioned generally within the center of the top surface 114. The cavity 120 may be defined by a cavity edge 128 at or near top surface 114, a cavity wall 122 that extends downward from the cavity edge 128 towards a bottom cavity edge 126, and a cavity floor 124 positioned near a bottom of the cavity wall 122.

As shown in FIG. 1, the cavity 120 may define a cavity volume that is generally described by the cross-sectional area of the cavity 120 (parallel to the cavity floor 124) and the depth of the cavity 120 from the cavity floor 124 to the top surface 114.

The cavity floor 124 may define a plurality of structures, cell dividers 136, extending upward from the cavity floor 124 toward the top surface 114. In many embodiments, such as that shown in FIG. 1, the cell dividers 136 may be linear with most dividers 136 defining two generally planar surfaces perpendicular to the cavity floor 124. As shown in the embodiment in FIG. 1, the cell divider 136 may extend between opposite walls of the cavity, and help to define a plurality of segments (e.g., 130a, b). Each cell divider segment may help to define an individual cell formed between intersecting segments. Each segment within an individual cell may define a wall 132 of the cell. In accordance with various embodiments, one or more of the walls including the cavity wall, the divider wall or the like may be generally perpendicular which include a tapered angle suitable to allow for the release of the dried contents therein.

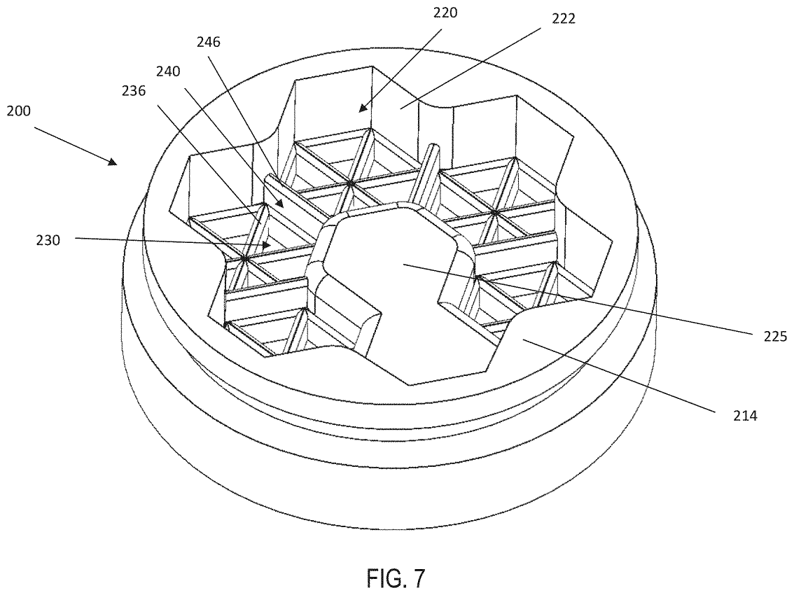

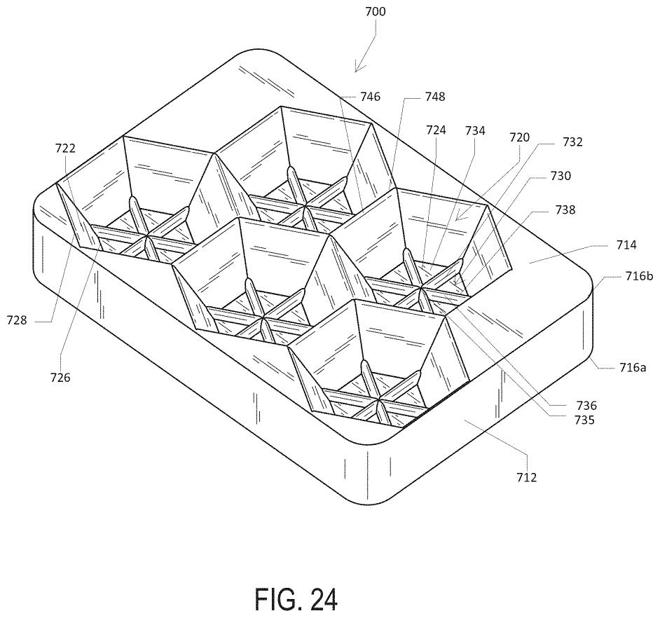

Individual cells may be various shapes and sizes. The cells of FIG. 1 are square-shaped, but may be selected from triangular (e.g., see FIGS. 7, 16 and 20), rectangular or square (e.g., see FIGS. 1, 8 and 12), wedge, hexagonal (e.g., see FIGS. 7 and 24), and octagonal, or any other suitable shape. In most embodiments, the cells are shapes that fit together in regular and repeating patterns.

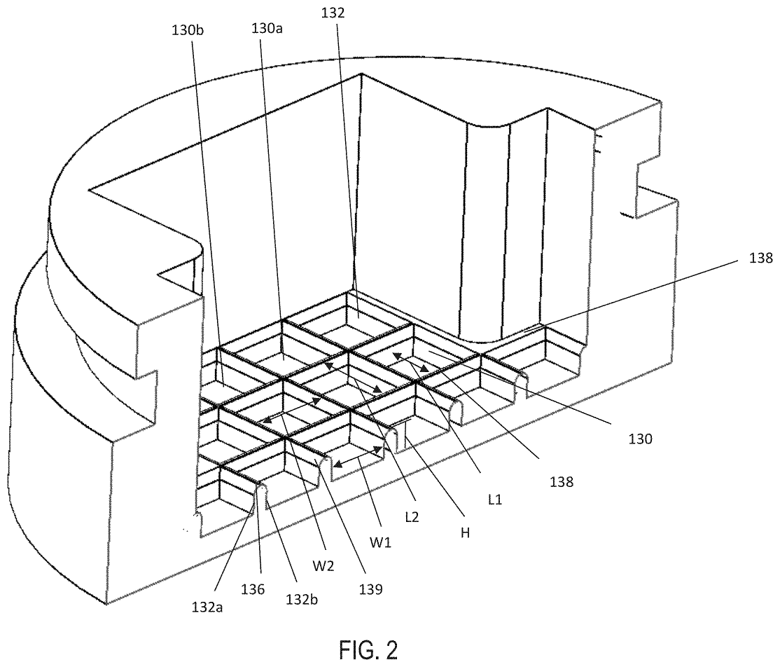

Most cell dividers 136 of the disclosed container 100 define a first generally planar surface perpendicular to the cavity floor 124, and a second planar surface that is also generally perpendicular to the cell floor 134. In some embodiments, the first and second planar surfaces may be angled such that they intersect at or near a divider top or apex 138, which is positioned away from the cavity floor 124. In some embodiments, as seen in FIG. 2, the divider 136 may define a shoulder structure 139 positioned between the apex 138 and the planar surface of the wall (e.g. 132a or 132b). The first 132a and second 132b planar surfaces help define the divider 136 and separate a first cell 130a from a second, adjacent cell 130b.

The cell divider 136 may be positioned at or near the cavity wall 122, the structure may be referred to as an edge divider or shoulder 138. The edge divider 138 may define a shape that is similar to that of other cell dividers 136, but comprises only one generally planar surface perpendicular to the cavity floor 124. In most embodiments, the edge divider 138 may define a shape that is roughly half that of a cell divider 136. In this way, even the edge cells can have a similar shape and dose content to interior cells.

In accordance with one embodiment, as illustrated in FIGS. 1, 2, and 4, the cells 130 have a rectangular shape that defines separate dose volumes of the dried contents within the container 100. In one example of this, the cells are square and are uniform throughout. However, in other embodiments, the cells can have other shapes and be configured to define a hierarchy of dose volumes. For example, as illustrated in FIG. 7, the container 200 includes primary 230, secondary 240, and tertiary cells (the tertiary cell being the cavity 220 as a whole.) The primary cells 230 may include the cells having the smallest dose volume. The primary cells 230 may be divided by primary cell dividers 236. The secondary cells 240 include a plurality of primary cells 230, but are still separated from other secondary cells 240 via secondary cell dividers 246. The secondary cell dividers 246 may have a height (e.g., from the cell floor towards the top surface 214) that is greater than the height of the primary cell dividers 236. As shown as an example, there are 5 secondary cells 240 with each secondary cell 240 having six primary cells 230 therein. The tertiary cell can be the entire cavity 220 as shown, or in other embodiments there can be a plurality of tertiary cells in a larger cavity (e.g., with tertiary cell dividers having a height greater than the secondary and primary cell dividers). In this way, the container 200 provides multiple tiers of identifiable doses upon the release of the contents, such as the three shown in FIG. 7. Additionally or alternatively, as shown in the embodiment of FIG. 7, the different tiers of cells can also have different shapes. As shown, the primary cells 230 are triangular, but as the contents of the primary cells 230 pours over the dividers 236, the secondary cells 240 are filled, which are hexagons as shown. As the contents of the secondary cells 240 pours over the dividers 246, a new shape is formed in the cavity 220.

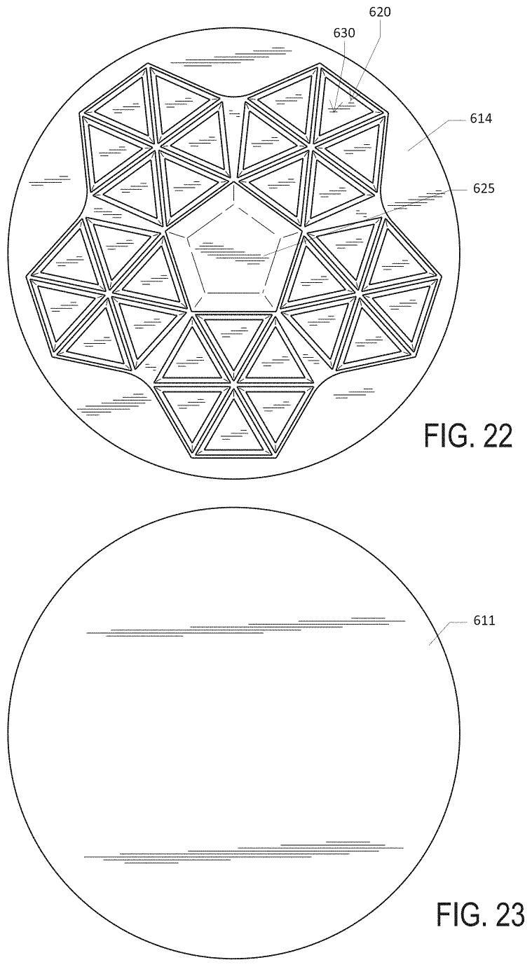

While in some embodiments, the entire cavity may be utilized for contents, in some embodiments, one or more of the primary cells or the secondary cells may be occupied by a marking surface 225, 625, as shown in FIGS. 7 and 22, respectively. The marking surface 225, 625 may be suitable to identify the contents of the container or the dosing capabilities of the container.

Referring now in more detail to FIG. 2, individual cells 130 define a height (h) that is measured from the cell floor 134 to the divider apex or top 138, and a cell area. In the embodiment of FIG. 2, the cell 130 has a first area, the cell floor area, measured at or near the cell floor 134 and defined by a first width (W1) and a second width (L1), and a second area, the cell apex area, defined by a first width (W2) and a second width (L2). In some embodiments, the cell floor area and the cell apex area are equivalent, and in other embodiments, the cell apex area may be greater than the cell floor area—for example where the first and second divider surfaces angle toward each other and meet at the apex. In some embodiments, the cell 130 may have an intermediate area positioned between the cell floor 134 and the cell apex 138. The volume of a given cell 130 may be measured by multiplying the average area of the cell 130 (which may be an average of the cell floor area and the cell apex area, or some fraction of the cell floor area), and the height (h) of the cell 130. As will be apparent to those of skill in the art, a round or circular-shaped cell 130 may have an area defined by the average radius of the circle.

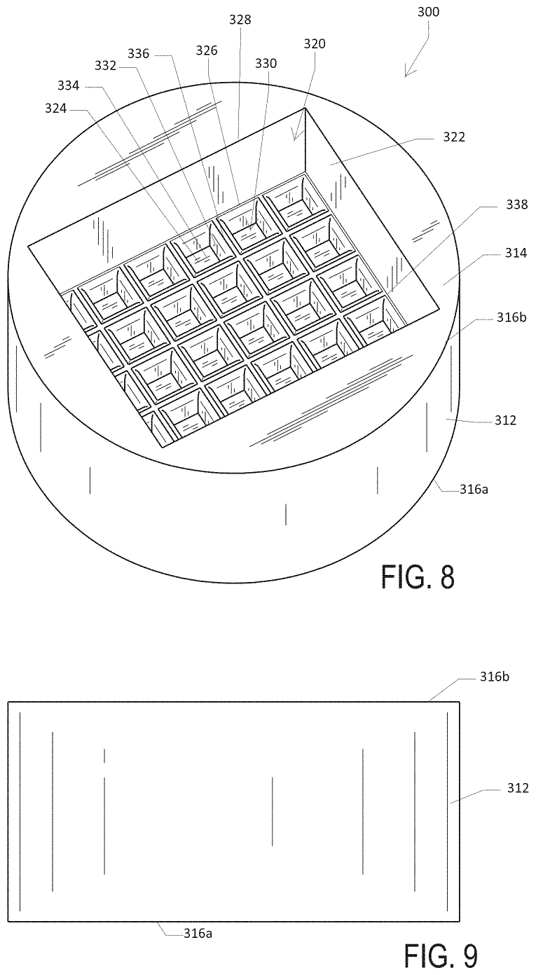

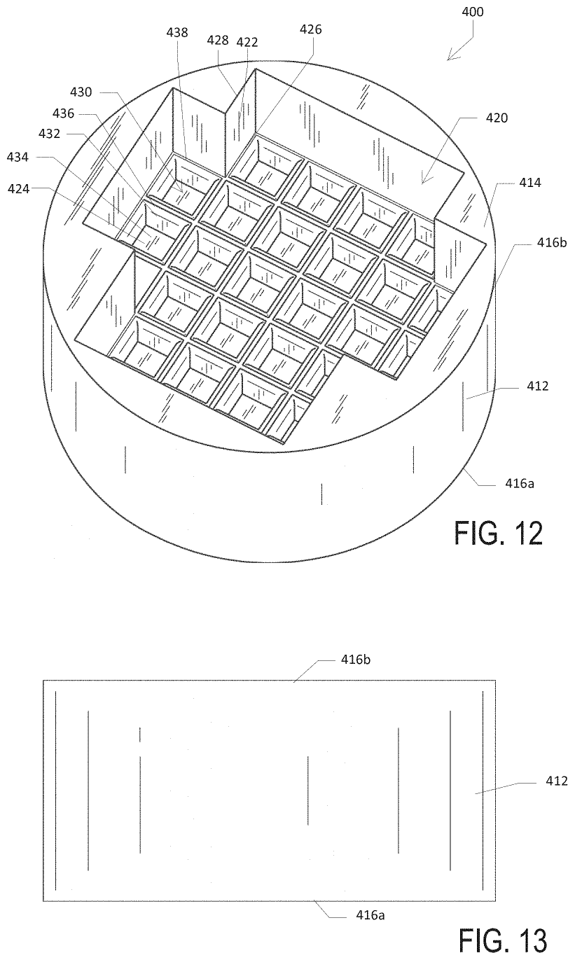

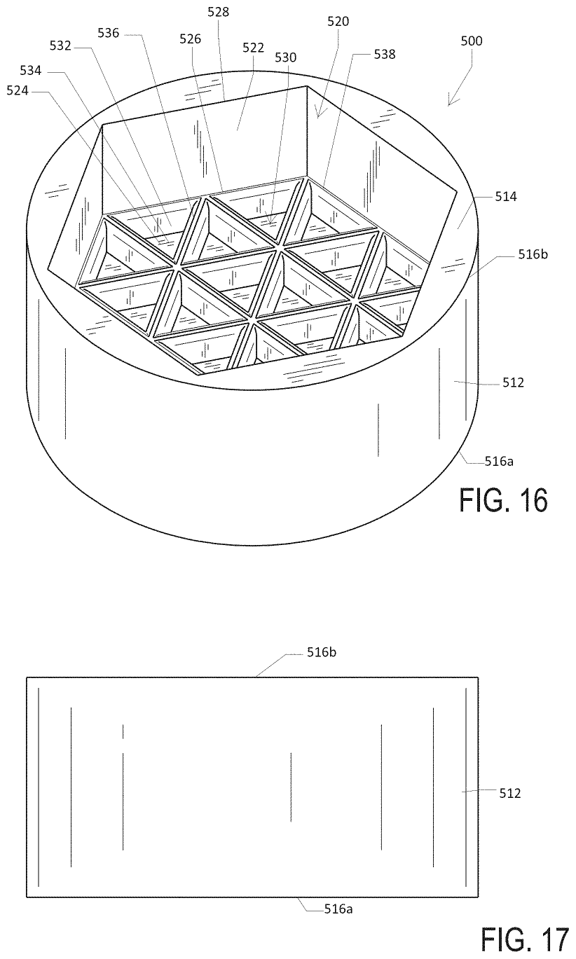

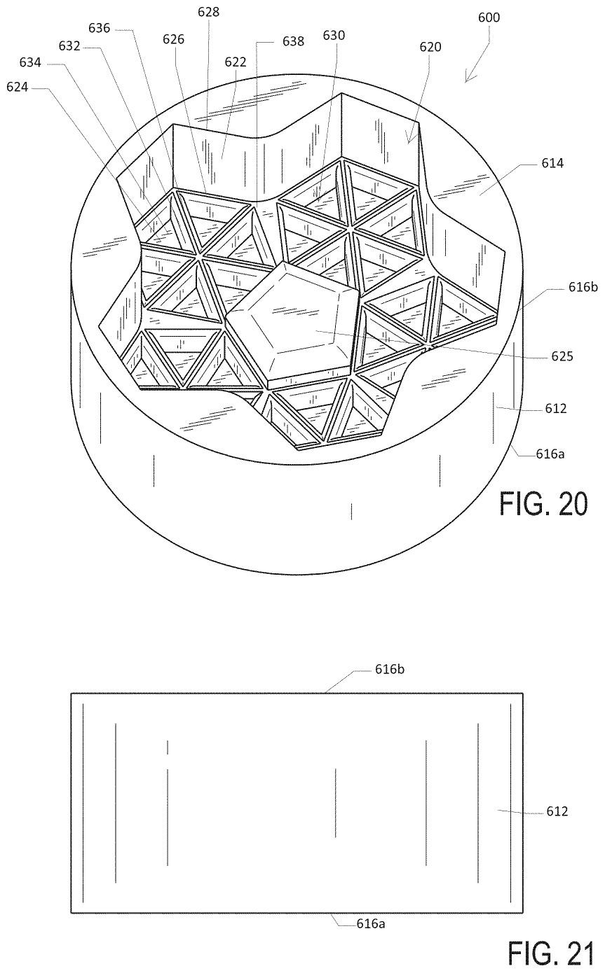

FIGS. 8-11, 12-15, 16-19, and 20-23 show additional embodiments of containers 300, 400, 500, and 600, respectively. As shown, the containers 300, 400, 500, and 600 form a generally circular or cylindrical shape and have an exterior or outer surface 312, 412, 512, 612, respectively. The outer surface 312, 412, 512, 612 extends from a respective base or bottom edge 316a, 416a, 516a, 616a to a respective upper or top edge 316b, 416b, 516b, 616b. As shown in FIGS. 11, 15, 19, and 23, the underside of the respective container 300, 400, 500, 600 may define a respective bottom surface 311, 411, 511, 611 for use in resting the container on a receiving surface. In most embodiments, the bottom surface 311, 411, 511, 611 may be generally planar. At or near the top edge 316b, 416b, 516b, 616b may be an upper or top surface 314, 414, 514, 614. Like the bottom surface 311, 411, 511, 611 the top surface 314, 414, 514, 614 may be generally planar, but may define an indentation or cavity 320, 420, 520, 620. As shown, the cavity 320, 420, 520, 620 may be defined by an upper cavity edge 328, 428, 528, 628 at or near the top surface 314, 414, 514, 614, a cavity wall 322, 422, 522, 622, a lower cavity edge 326, 426, 526, 626, and a cavity floor 324, 424, 524, 624. The upper cavity edge 328, 428, 528, 628 may be defined by the intersection between the cavity wall 322, 422, 522, 622 and the top surface 314, 414, 514, 614. As shown, the upper cavity edge 328, 428, 528, 628 may form a rectangular shape (FIG. 8), a t-shape or cross shape (FIG. 12), a hexagonal shape (FIG. 16), or a star-like shape (FIG. 20) defined within the top surface 314, 414, 514, 614. The cavity floor 324, 424, 524, 624 may be positioned near a bottom of the cavity wall 322, 422, 522, 622. The lower cavity edge 326, 426, 526, 626 may be defined by the intersection between the cavity wall 322, 422, 522, 622 and a cell divider apex or top 338, 438, 538, 638. The cavity wall 322, 422, 522, 622 may extend downward from the upper cavity edge 328, 428, 528, 628 to the lower cavity edge 326, 426, 526, 626 and to the cavity floor 324, 424, 524, 624. The cavity wall 322, 422, 522, 622 may extend perpendicular from the cell divider apex 338, 438, 538, 638 or cavity floor 324, 424, 524, 624, or, alternatively at an angle, forming a slanted wall such that the upper cavity edge 328, 428, 528, 628 has a larger dimension than the lower cavity edge 326, 426, 526, 626.

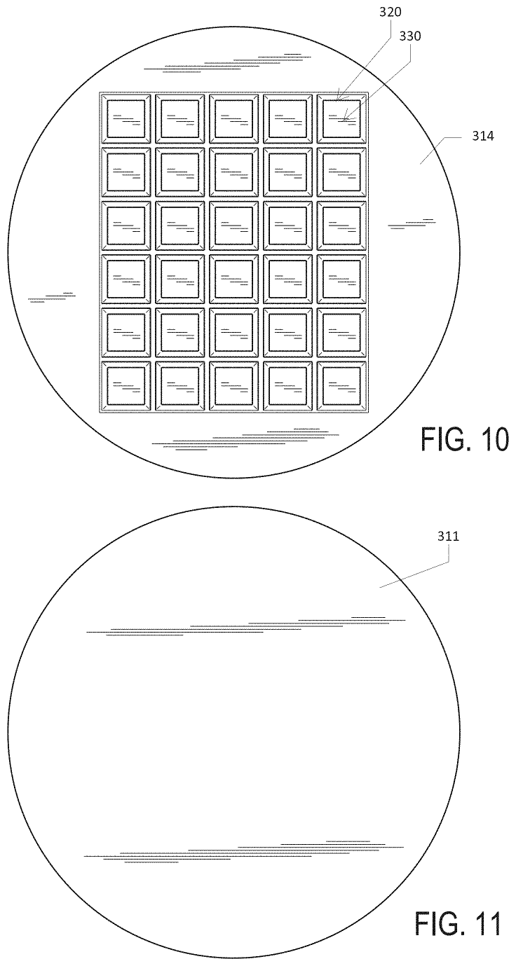

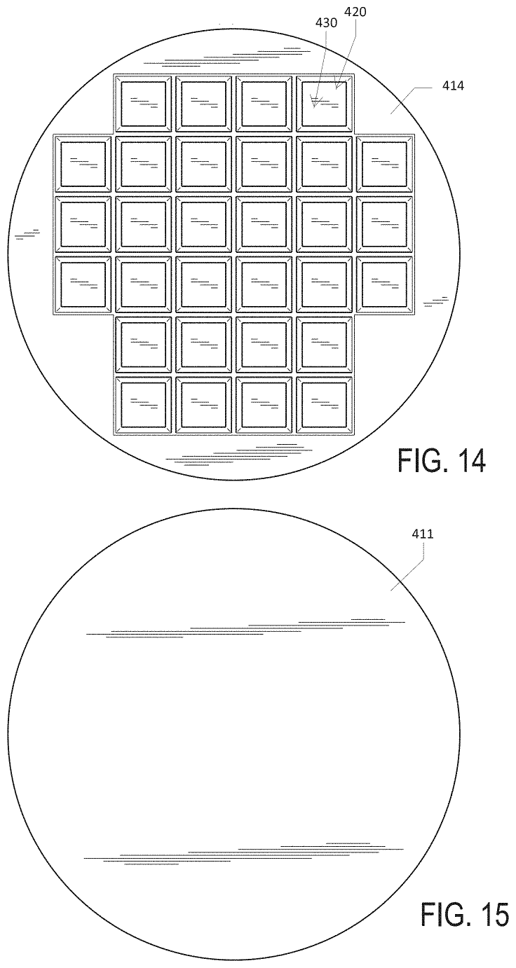

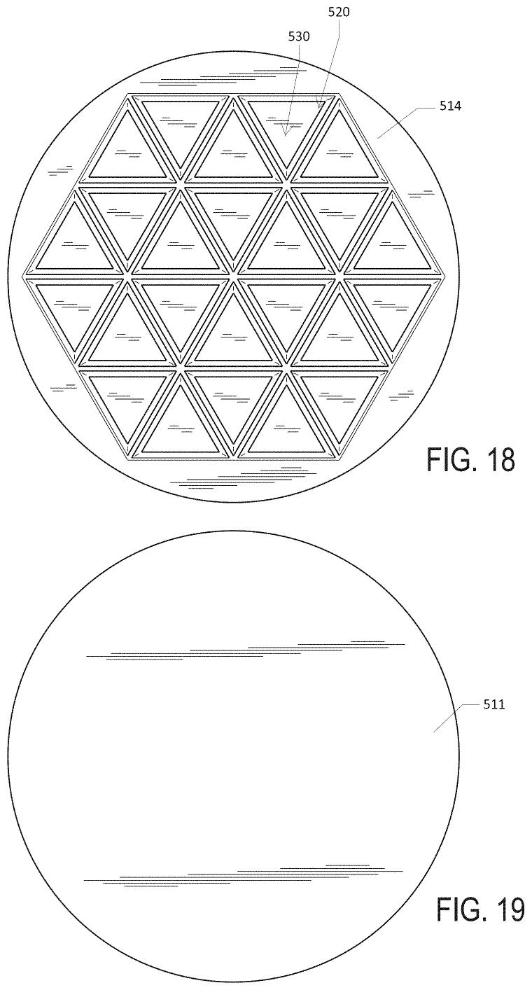

As shown in FIGS. 10, 14, 18, and 22, the cavity 320, 420, 520, 620 may include a plurality of cells 330, 430, 530, 630 that define a plurality of identifiable volumes (e.g., either generally uniform or variable). As shown in FIG. 10, the cavity 320 includes 30 cells 330; as shown in FIG. 14, the cavity 420 includes 30 cells 430; as shown in FIG. 18, the cavity 520 includes 24 cells 530; as shown in FIG. 22, the cavity 620 includes 30 cells 630. The cells 330, 430, 530, 630 are separated by cell dividers 336, 436, 536, 636 extending upward from the cavity floor 324, 424, 524, 624 part of the way up the cavity walls 322, 422, 522, 622 toward the top surface 314, 414, 514, 614. As shown in FIGS. 8, 12, 16, and 20, the cell dividers 336, 436, 536, 636 may be linear with most dividers 336, 436, 536, 636 defining two generally planar surfaces perpendicular to the cell divider apex 338, 438, 538, 638. The cells 330, 430, 530, 630 are defined by intersecting cell dividers 336, 436, 536, 636, each forming a cell wall 332, 432, 532, 632 of the cell 330, 430, 530, 630, and a cell floor 334, 434, 534, 634. As shown in FIGS. 8 and 12, the cells 330, 430 are defined by four intersecting cell dividers 336, 436 forming four cell walls 332, 432 and a square shape for each cell 330, 430. As shown in FIGS. 16 and 20, the cells 530, 630 are defined by three intersecting cell dividers 536, 636 forming three cell walls 532, 632 and a triangular shape for each cell 530, 630.

In some embodiments, the cells may have a shape that is compatible with or conforms to the shape of the cavity such that uniform cells fill the cavity space. For example, as shown in FIGS. 10, 14, and 18, the cells 330, 430, 530 fill the space of the respective cavity 320, 420, 520. In these embodiments, the cell dividers 336, 436, 536 are of uniform size (e.g., have a uniform width). In other embodiments, the cells may have a shape that is not compatible with and does not conform to the shape of the cavity such that uniform cells do not fill the cavity space. For example, as shown in FIGS. 4 and 22, the cells 130, 630 do not fill the space of the respective cavity 120, 620. In these embodiments, the cell dividers 136, 636 are not of uniform size and instead fill the additional space within the cavity 120, 620. For example, as shown in FIG. 1, the cavity wall 122 curves such that the rectangular cells 130 do not align with the cavity wall 122. The cell dividers 136 fill the additional space between the curved cavity wall 122 and the cell wall 132. As another example, as shown in FIG. 20, some adjacent cells 630 are angled away from one another due to curvature of the cavity wall 622. In this example, the cell dividers 636 fill the additional space between adjacent cell walls 632 that diverge away from one another.

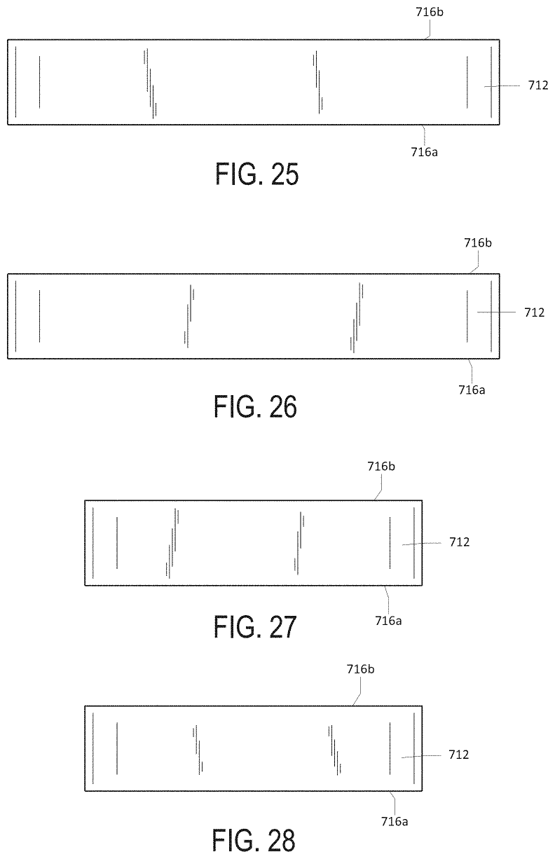

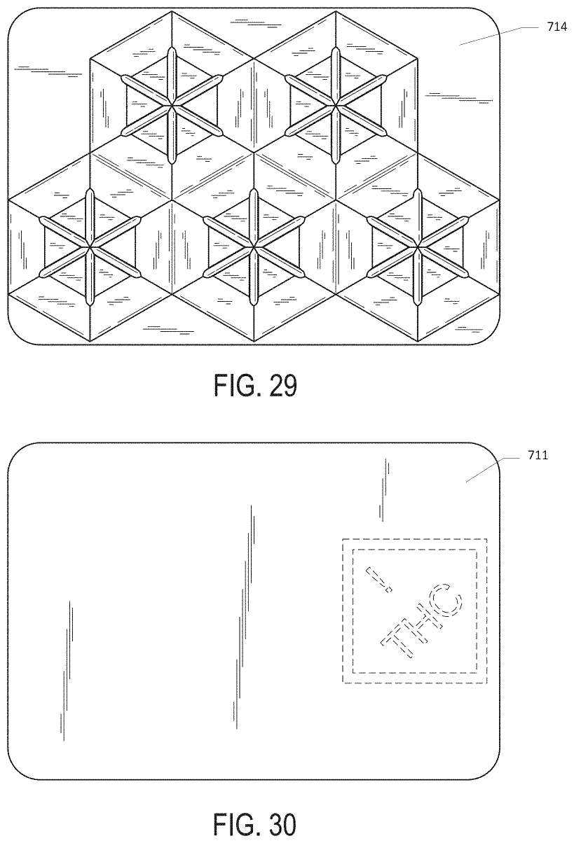

In some embodiments, a top surface of a container of the present disclosure may define a plurality of cavities. For example, FIGS. 24-30 show an embodiment of a container 700 including a plurality of cavities 720. As shown, the container 700 forms a generally rectangular shape and has an exterior or outer surface 712. The outer surface 712 extends from a base or bottom edge 716a to an upper or top edge 716b. As shown in FIG. 30, the underside of the container 700 may define a bottom surface 711 for use in resting the container 700 on a receiving surface (not shown). In most embodiments, the bottom surface 711 may be generally planar. At or near the top edge 716b may be an upper or top surface 714. Like the bottom surface 711, the top surface 714 may be generally planar, but may define a plurality of indentations or cavities 720. In the depicted example, the container 700 includes 5 cavities 720, however, more or less cavities are contemplated. As shown, each cavity 720 may be defined by an upper cavity edge 728 at or near top surface 714, a cavity wall 722 (e.g., having six surfaces), a lower cavity edge 726, and a cavity floor 724. The upper cavity edge 728 may be defined by the intersection between the cavity wall 722 and the top surface 714. As shown, the upper cavity edge 728 may form a hexagonal shape defined within the top surface 714. The cavity floor 724 may be positioned near a bottom of the cavity wall 722. The lower cavity edge 726 may be defined by the intersection between the cavity wall 722 and the cavity floor 724. The cavity wall 722 may extend downward from the upper cavity edge 728 to the lower cavity edge 726. In the depicted embodiment, the cavity wall 722 extends downward from the upper cavity edge 728 to the lower cavity edge 726 at an angle, forming a slanted wall such that the upper cavity edge 728 has a larger dimension than the lower cavity edge 726 (e.g., forms a greater perimeter around the cavity 720); however, it is also contemplated that the cavity wall 722 may be perpendicular to the cavity floor 724 such that the upper cavity edge 728 and lower cavity edge 726 have the same dimension. In various examples, the cavities 720 may be positioned adjacent one another with a cavity divider 746 positioned between and separating adjacent cavities 720. As shown, the cavity walls 722 of adjacent cavities 720 may be angled such that they intersect at or near a cavity divider top or apex 748, which is positioned away from the cavity floor 724.

As shown, each cavity 720 may include a plurality of cells 730 that define a plurality of identifiable volumes (e.g., either generally uniform or variable). In the depicted embodiment, each cavity 720 includes 6 cells 730; however, more or less cells are contemplated. As shown, the container 700 includes 30 cells 730 total. Each cavity 720 is evenly divided, such that the cells 730 are uniformly shaped. The cells 730 are divided by cell dividers 736 extending upward from the cavity floor 724 part of the way up the cavity walls 722 toward the top surface 714. In many embodiments, such as that shown in FIG. 24, the cell dividers 736 may be linear with most dividers 736 defining two generally planar surfaces perpendicular to the cavity floor 724. As shown in the embodiment in FIG. 24, a cell divider 736 may extend from a center intersection point 735 where a plurality of cell dividers 736 intersect to the cavity wall 722. Each cell 730 is defined by intersecting cell dividers 736 and the cavity wall 722, each forming a cell wall 732 of the cell 730, and a cell floor 734. In the depicted embodiment, the two cell walls 732 formed by the cell dividers 736 are perpendicular to the cell floor 734, while the cell wall 732 formed by the cavity wall 722 forms an obtuse angle relative to the cell floor 734. The tapered angle of the cell wall 732 facilitates release of dried contents stored within the cell 730. As shown, the cells 700 form a triangular or wedge shape. In the embodiment depicted in FIG. 24, the cells 730 have a hierarchy of dose volumes. As illustrated, the container 700 includes primary cells 730 and secondary cells (the secondary cells being the cavities 720). In this example, the primary cells 730 have the smallest dose volume. The secondary cells include a plurality of primary cells 730, but are still separated from other secondary cells via cavity dividers 746. In this way, the container 700 provides two tiers of identifiable doses upon the release of the contents. As shown, the different tiers of cells have different shapes. As shown, the primary cells 730 are triangular, but as the contents of the primary cells 730 pours over the cell dividers 736, the secondary cells are filled, which are hexagons as defined by the cavity 720.

### Volume

In many embodiments, the container may be configured to hold a substance having a volume (V_{S}) that is greater than the sum of all individual cells (c1, c2, c3, c4 . . . cn) of the container (V_{C1-Cn}). In many embodiments, the substance may be dried to create a solid, such as a waxy or crystalline substance, and the dried substance may also have a volume, V_{Sd} that is greater than V_{C1-Cn}. In those embodiments, wherein the cells define a total collective volume that is less than that of the substance within the container, the excess volume may form a uniform layer above the level of the divider apex. As used herein, an equivalent volume may reflect a difference of less than 10% between two different volumes, for example less than 10%, 9%, 8%, 7%, 6%, 5%, 4%, 3%, or 2%, and greater than about 1%, 2%, 3%, 4%, 5%, 6%, 7%, 8%, and 9%.

In several embodiments, the container may be configured to hold two, three, or more different volumes. For example, primary cells 230, 730 may each define a first volume, secondary cells 240, 720 may each define a second volume, and optionally, tertiary cells 220 may each define a third volume. In some embodiments, the third volume is greater than the second volume, and the second volume is greater than the first volume. In the example shown in FIG. 7, the second volume is based on the height of the secondary cell dividers 246. In the example shown in FIG. 24, the second volume is based on the height of the cavity dividers 746. In several embodiments, the second volume (e.g., defined by each secondary cell 240, 720) may range between 50-200 mg, 75-250 mg, 100-300 mg, 200-250 mg, and the like. As one example, the second volume is 200 mg. In embodiments including tertiary cells, the third volume defined by a tertiary cell may vary, and may be broken up into the second volume to create a targeted dose.

### Molding

Each container includes an arrangement of cells having approximately equivalent volumes. The cells are configured to define equivalent volumes, such that active ingredients, in liquid form, can be dispensed into the container, and, when solidified, an equivalent amount of active ingredient can be removed from each volume. In most embodiments, the dividers are intended to create a scoring or etching pattern in the solidified product, such that even if the volume of solid product is significantly greater than total volume of all cells, the user may break off individual, cell-shaped portions along the scoring lines. In embodiments lacking edge dividers, the cells located adjacent the cavity wall may be larger, or those cells may, alternatively, define doses with slightly less active ingredient. In accordance with some embodiments, the top of the cell wall or a portion thereof can extend high enough so that it is proud of the planer surface of the shatter. This enables a perforation to form in the shatter.

The polymer may be various materials. In some embodiments, the polymer comprises a polymeric siloxane, for example polysiloxane comprising a plurality of repeating units of siloxane. In most embodiments, the polymer is a silicone, for example a medical or food grade silicone known to those of skill in the art. The polymer may be cured or cross-linked to aid in solidifying the polymer. Thus, in many embodiments, the polymer may further include one or more cross-linking moieties connecting the polymer chains of, for example, siloxane.

### Cap

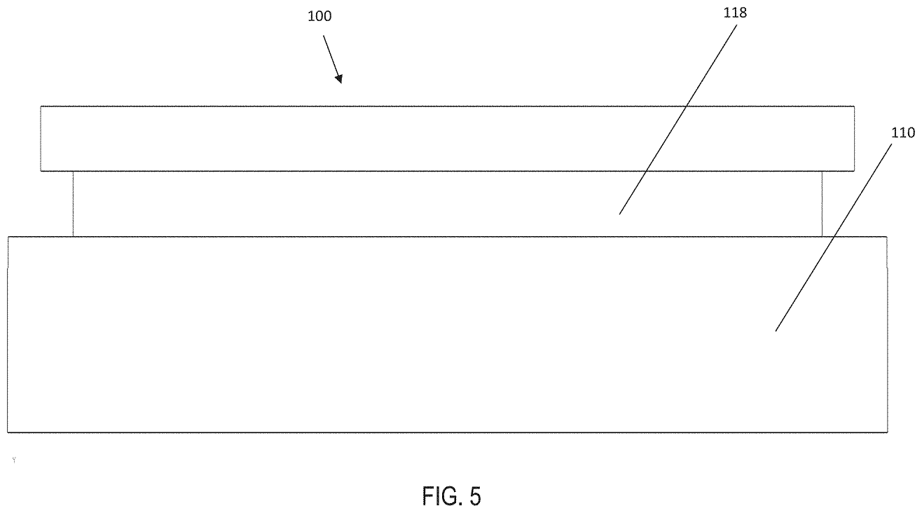

Turning then to the embodiment shown in FIGS. 1, 2, 5 and 6, it is seen that the container 100 is generally defined by the upper surface, outer surface 112, and cavity 120 formed in the upper surface. In many embodiments, the container 100 is associated with an integral, connected, or independent cap 160. In some embodiments, one or more of the container 100 or the cap may be designed with one or more structures that may aid in securing the cap to the container 100, so that the product within the cavity 120 does not spill out of the container 100 when it is turned upside down. In various embodiments, the structure provides a fastening mechanism to hold the cap 160 onto the container 100. Fastening mechanisms can include threads, detents, snaps, connectors or other suitable connection structures. In one example, the container 100 may include a channel 118 that extends around the body 110. More specifically, the channel may extend around the outer surface 112. The cap 160 can include an opposing annular ridge 162 or similar structure suitable to be received into the channel 118, allowing the two structures to form a releasable connection between the cap 160 and the container 100.

### Examples

In one example, as shown in FIG. 7, the secondary cells 240 of the mold (e.g., each having six primary cells 230) include size relationships as indicated in the table below.

| Number of | Calculated | Calculated | |
|---|---|---|---|
| Cells | SA\hat{ }2 | Volume \hat{ }3 | |
| Measured | (sq. in.) | (cubic in.) | SA/V |
| 1 | 0.614 | 0.017 | ≈36⅛ |
| 5 | 3.07 | 0.085 | |

In another example, as shown in FIG. 24, the secondary cells of the mold (i.e., the cavities 720 each having six primary cells 730) include size relationships as indicated in the table below.

| Number of | Calculated | Calculated | |
|---|---|---|---|
| Cells | SA\hat{ }2 | Volume \hat{ }3 | |
| Measured | (sq. in.) | (cubic in.) | SA/V |
| 1 | 0.912 | 0.044 | ≈20¾ |
| 5 | 4.56 | 0.22 | |

The surface area to volume (SA/V) ratios depicted in the above examples are illustrative only and other SA/V relationships for the primary, secondary, and/or tertiary cells are contemplated. For example, the secondary cells may each have a surface area to volume ratio that ranges between 15-45, 20-40, 30-37, is about 20, is about 36, and the like.

### Method of Apportioning a Solid Product

Disclosed herein is a method of apportioning a product. In most embodiments, the product may be added to the container in liquid form and may solidify within the container. In some embodiments, the product may be liquid at high temperature and may solidify at ambient temperatures. In some embodiments, the product may solidify when a volatile solvent is removed. In some embodiments, removal of a volatile solvent may be removed by methods including increasing the temperature of the product and lowering the ambient pressure. In some embodiments, the product may be subjected to partial or complete lyophilization.

The disclosed methods are useful in creating a separable solid pharmaceutical, herbal, or nutraceutical product having individual portions that comprise similar amounts of an active ingredient. In some embodiments, the active ingredient may be an essential oil, terpene, terpenoid, cannabinoid (e.g., cannabinol), or the like. In some embodiments, the active ingredient may possess medicinal or homeopathic properties. In many embodiments, the active ingredient may be vaporized, volatilized, or atomized prior to administration.

The disclosed methods may include adding a liquid product containing an active ingredient to the container. The volume of liquid product added to the container is generally greater than the sum of the volumes of all cells 130, 230, 330, 430, 530, 630, 730 within the container 100, 200, 300, 400, 500, 600, 700. In most embodiments, the liquid product may form a sufficiently planar liquid surface within the container. In most embodiments, the planar liquid surface is at a distance from the floor 124, 224, 324, 424, 524, 624, 724 of the container 100, 200, 300, 400, 500, 600, 700 that is greater than the distance from the floor 124, 224, 324, 424, 524, 624, 724 to the cell divider top 138, 238, 338, 438, 538, 638, 738.

The disclosed methods may include allowing the liquid product to solidify within the container. In some embodiments, as the liquid product solidifies, the volume of the liquid product is reduced. In other embodiments, the volume of the liquid product in the container is not substantially reduced. In those embodiments, wherein the volume of the liquid product is greater than the volume of the solidified product, the solid product may define a planar surface at a distance from the container floor 124, 224, 324, 424, 524, 624, 724 that is less than that of the distance of the liquid planar surface from the container floor 124, 224, 324, 424, 524, 624, 724. In most of these embodiments, the distance from the container floor 124, 224, 324, 424, 524, 624, 724 to the solid planar surface is greater than that of the container floor 124, 324, 424, 524, 624, 724 to the top of the cell divider 138, 238, 338, 438, 538, 638, 738.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

1. A polymeric container for apportioning an active ingredient, the container comprising:
a cavity defined by a cavity floor and a plurality of cavity walls; and
two or more cells positioned within the cavity, the cells comprising a cell floor and at least one cell divider extending from the cell floor to a cell divider top, wherein each cell of the two or more cells defines a volume defined by a cross-sectional area of the respective cell and the distance from the cell floor to the cell divider top of the respective cell.

2. The container of claim 1, wherein the container cavity comprises between 5 and 30 cells.

3. The container of claim 2, wherein each of the between 5 and 30 cells defines a volume that is equivalent.

4. The container of claim 1, wherein the two or more cells defines a shape selected from a circle, triangle, square, hexagon, and octagon.

5. The container of claim 1, wherein the two or more cells define different shapes and equivalent volumes.

6. The container of claim 1, wherein the at least one cell divider extends from the cell floor to the cell divider top at a tapered angle suitable to allow for the release of dried contents containing the active ingredient therein.

7. The container of claim 1, wherein the volume defined by each of the two or more cells is sized to hold an equivalent amount of the active ingredient.

8. The container of claim 1, wherein the two or more cells define a primary dose volume and the cavity defines a secondary dose volume, wherein the secondary dose volume is filled when the active ingredient pours over the at least one cell divider.

9. The container of claim 8, wherein the primary dose volume has a first shape defined by the two or more cells and the secondary dose volume has a second shape defined by the cavity, and the first and second shapes are different.

10. A method of apportioning a liquid, comprising the following steps:
adding a volume of a liquid to a polymeric container having a cavity defined by a container floor and a plurality of container walls, and two or more cells positioned within the cavity, the cells comprising a cell floor and at least one cell divider extending from the cell floor to a cell divider top, wherein each cell defines a volume defined by a cross-sectional area of the respective cell and the distance from the cell floor to the cell divider top of the respective cell, wherein the volume of the liquid is greater than the sum of the volumes defined by the two or more cells;
allowing the liquid to form a planar surface generally positioned further from the cell floor than the level of the cell wall divider;
treating the liquid in the container to aid in transitioning the liquid to a solid; and
allowing the liquid to form a solid at room temperature.

11. The method of claim 10, wherein the treating step removes a volatile solvent.

12. The method of claim 10, wherein the treating step includes increasing the ambient temperature or decreasing the ambient pressure of the container.

13. The method of claim 10, wherein the active ingredient is selected from an essential oil, terpene, or cannabinoid.

14. The method of claim 10, wherein the active ingredient is cannabinol.

15. A container for apportioning equivalent doses of an active ingredient, the container comprising:
a plurality of primary cells, the primary cells defined by a primary cell wall and a primary cell floor, wherein
each primary cell defines a first shape and an equivalent first volume, and
adjacent primary cells are separated by a primary cell divider; and
a plurality of secondary cells, the secondary cells defined by a secondary cell wall and a secondary cell floor, wherein
each secondary cell defines a second shape and an equivalent second volume, and
adjacent secondary cells are separated by a secondary cell divider;
wherein the plurality of primary cells are positioned within the plurality of secondary cells and the secondary cell divider has a height greater than the primary cell divider.

16. The container of claim 15, wherein the plurality of secondary cells are cavities defined within a top surface of the container.

17. The container of claim 15, wherein the first shape and the second shape are different.

18. The container of claim 17, wherein the first shape is a triangle and the second shape is a hexagon.

19. The container of claim 15, wherein the first volume provides a smaller identifiable dose of the active ingredient than the second volume.

20. The container of claim 15, wherein a portion of the primary cell wall is defined by a portion of the secondary cell wall, and the secondary cell wall extends from the secondary cell floor at an obtuse angle.
